(19) United States
(12) Patent Application Publication
(10) Pub. No.: US 2025/0064546 A1
(43) Pub. Date: Feb. 27, 2025

(54) ANATOMIC SURFACE AND FIDUCIAL REGISTRATION WITH AN INTRA-OPERATIVE 3D SCANNER

(71) Applicant: CAIRA Surgical, New York, NY (US)

(72) Inventors: Morteza MEFTAH, New York, NY (US); Gordon L. GOODCHILD, West Palm Harbor, FL (US)

(21) Appl. No.: 18/941,844

(22) Filed: Nov. 8, 2024

Related U.S. Application Data
(63) Continuation-in-part of application No. 17/719,997, filed on Apr. 13, 2022.
Application No. 17/719,997 is a division of application No. 16/254,220, filed on Jan. 22, 2019, now Pat. No. 11,351,007.
(60) Provisional application No. 62/620,448, filed on Jan. 22, 2018.
(60) Provisional application No. 63/597,027, filed on Nov. 8, 2023.

(51) Int. Cl. A61B 90/00; A61B 17/15; A61B 34/10

(52) U.S. Cl. CPC A61B 90/37; A61B 17/155; A61B 17/157; A61B 34/10; A61B 2034/107; A61B 2090/366; A61B 2090/373

(57) ABSTRACT

Aspects of the present disclosure include surgical systems and methods for modifying an existing 3D model of a region of anatomic interest to include features intra-operatively scanned by a 3D scanner. In one aspect, the systems and methods include operations for: receiving scan data from an intra-operative scan of a bone surface in the region of anatomic interest, where the scan data includes a fiducial placed by a surgeon; generating an intra-operative 3D model that includes a 3D representation of the fiducial; registering the intra-operative 3D model with a pre-operative 3D model of the region of anatomic interest; modifying the pre-operative 3D model to include the fiducial at a location according to the intra-operative 3D model; and providing the modified pre-operative 3D model to a surgical navigation system that tracks objects in the region of anatomic interest based on the location of the fiducial.

### RELATED APPLICATION DATA

This application is a Continuation-in-Part of pending U.S. patent application Ser. No. 17/719,997, filed 13 Apr. 2022, which is a Divisional of U.S. patent application Ser. No. 16/254,220, filed 22 Jan. 2019, (now U.S. Pat. No. 11,351,007), which claims the benefit and priority of U.S. Provisional Patent Application Ser. No. 62/620,448, filed 22 Jan. 2018, and entitled “Surgical System With Intra-Operative 3D Scan,” all of which are hereby incorporated by reference herein in their entireties. This application also claims the benefit and priority of U.S. Provisional Patent Application Ser. No. 63/597,027, filed 8 Nov. 2023, which is also hereby incorporated by reference herein in its entirety.

### FIELD OF THE INVENTION

The present invention generally relates to the field of surgical systems. In particular, the disclosure is directed to surgical systems with intra-operative 3d scanners and surgical methods using the same, for example, for intra-operative registration and subsequent tracking of a surgeon-placed fiducial.

### BACKGROUND

Joint replacement surgery has become an ever-increasing area of surgical procedures. It has been reported that more than 7 million Americans living with a hip or knee replacement. A 2017 report of American Joint Registry shows 860,080 procedures from 654 institutions and 4,755 surgeons, representing a 101% increase in procedures from the year prior. Kurt et al in an article in the Journal of Bone and Joint Surgery estimate that 700,000 knee replacement procedures are performed annually in the US, and this number is projected to increase to 3.48 million procedures per year by 2030. The current annual economic burden of revision knee surgery is $2.7 billion for hospital charges alone, according to Bhandari et al in Clinical Medical Insights: Arthritis and Musculoskeletal Disorders (2012). By 2030, assuming a 5-fold increase in the number of revision procedures, this economic burden will exceed $13 billion annually (Bhandari et al). Adding to the number of the procedures and the economic burden is the fact that of the total knee replacements per annum, around 3% need to be revised for malposition/malalignment. This constitutes more than 21,000 cases a year of suffering patients who need to undergo a revision surgery.

Currently there are two ways of performing a knee replacement, either with conventional instruments or computer aided surgery. Most cases in the United States are performed using conventional instruments. This technique involves using intra- or extra-medullary rods to reproduce the anatomic axes. For the proximal tibial cut, an extramedullary rod is conventionally used. The distal portion of the rod is clamped around the ankle and the tibia is cut perpendicular to the anatomical axis. For the distal femoral cut, an intra-medullary rod is also conventionally used. The femur is drilled to accept the rod and then the distal femur is arbitrarily cut at 5 degrees, with a range of 3 to 7 degrees. The rotational position of the femur and tibia is mostly achieved by visually identifying anatomical landmarks or some form of gap balancing methods. The drawbacks to conventional alignment systems include difficulty with identifying the anatomic landmarks, (e.g., the lateral epicondyle, lateral condyle, medial epicondyle, medial condyle, or the intercondylar fossa, etc., of a femur), intra-operatively as well as the assumption of standard anatomic relationships, which may not always be consistent or accurate across all patients.

Computer-assisted surgery (CAS) was developed to help achieve more customized, precise, and repeatable techniques. CAS, and especially orthopedic CAS, typically employs an image-based navigation system (a.k.a. an optical tracking system) that requires intra-operative registration of anatomical landmarks to a pre-operative three-dimensional (3D) model of the region or area of anatomical interest (e.g., the bones of a knee joint), where the pre-operative 3D model is created from pre-operative images, such as pre-operative MRI images. Typically, this registering process is long, tedious, and involves multiple steps with a handheld pointer probe that is optically tracked by the navigation system as it is positioned by the surgeon to identify anatomic landmarks.

For example, typical conventional computer-assisted orthopedic surgery requires manual acquisition and registration of bony landmarks using a CAS pointer probe or the like, and the attachment of optical trackers to bones for calibration and tracking. The trackers are usually outside of the incision and must be well fixed to the bone because any movement can lead to errors in the acquisition of data for the CAS navigation system. Among other drawbacks, the manual acquisition of anatomic landmarks by palpation is time-consuming, error-prone—its accuracy is surgeon-dependent, and it is not consistently reproducible.

Other drawbacks include that the surgeons must be specially trained in multiple manual registration techniques using different probes so to be able to employ the manual registration techniques required by each different CAS system. These drawbacks detrimentally prolong the time in surgery, increase the incidents of malpositioning of implants (e.g., knee joint implants), (which can lead to instability, pain, decreased range of motion, and implant loosening), and increase the cost of orthopedic operations.

### SUMMARY OF THE DISCLOSURE

In one aspect, the present disclosure is directed to systems, methods, and computer program products for modifying a 3D model of an area of anatomic interest. The systems, methods, and computer program products may have features that include: a fiducial that is attached to a bone surface in the area of anatomic interest; an intra-operative three-dimensional (3D) scanner; and a computer that is connected to the intra-operative 3D scanner. The computer may perform a process or operations that may include: receiving, from the intra-operative 3D scanner, scan data from an intra-operative scan of the bone surface, wherein the scan data includes data representing the fiducial; generating, from the scan data, an intra-operative 3D model that includes a 3D representation of the fiducial; registering the intra-operative 3D model with a pre-operative 3D model of the area of anatomic interest; modifying the pre-operative 3D model to include the fiducial at a location according to the intra-operative 3D model; and providing the modified pre-operative 3D model to a surgical navigation system that tracks objects in the area of anatomic interest based on the location of the fiducial.

In another aspect, the present disclosure is directed to systems, methods, and computer program products in which the intra-operative 3D scanner may be a handheld laser 3D scanner. In another aspect, the present disclosure is directed to systems, methods, and computer program products in which the pre-operative 3D model may be created from MRI scan data.

In another aspect, the operations may include employing a machine learning model to identify an anatomical landmark in the intra-operative 3D model.

In another aspect, the fiducial is a plate or an anchor. In such aspects, the fiducial may also be attached to the bone surface at a predetermined location. In further such aspects, the predetermined location may be based on a preoperative image of the bone surface. In further such aspects, the preoperative image may be any of a computer tomography image, an ultrasound image, and/or a magnetic resonance image. In another aspect where the fiducial is a plate, the plate may include a bar code the encodes surgery-assisting information.

In another aspect, the operations may include analyzing the intra-operative 3D model to differentiate between different types of tissue. In such aspects, the analyzing may employ machine learning methods. In further aspects, the different types of tissue may include bone tissue and cartilage tissue.

In another aspect, fiducial may be attached intra-operatively without using a predetermined location on the bone surface, and the operations may further include analyzing the intra-operative 3D model to detect the location of the fiducial.

In yet another aspect, the operation for registering the intra-operative 3D model with a pre-operative 3D model may include aligning the intra-operative 3D model with the pre-operative 3D model using an iterative closest point (ICP) algorithm.

In yet another aspect, the operation for modifying the pre-operative 3D model to include the fiducial at a location according to the intra-operative 3D model may include combining the intra-operative 3D model with the pre-operative 3D model to produce the modified pre-operative 3D model. In such aspects, the modified pre-operative 3D model may include the fiducial from the intra-operative 3D model.

### BRIEF DESCRIPTION OF THE DRAWINGS

These features, aspects, and advantages of the present disclosure will become better understood with regard to the following description and accompanying drawings which illustrate exemplary features of the disclosure. However, it is to be understood that each of the features can be used in the disclosure in general, not merely in the context of the particular drawings, and the disclosure includes any combination of these features, where:

FIG. 1A is a schematic diagram of an example surgical system made in accordance with the present disclosure;

FIG. 1B shows a surgical light that includes a 3D scanner for use with the surgical system of FIG. 1A;

FIG. 1C is a representative 3D image of an anterior view of the distal end of a femur;

FIG. 1D is a functional block diagram of the computer of FIG. 1A;

FIG. 2 is a flow chart illustrating an example surgical method;

FIG. 3A is a representative 3D image of an anterior view of a distal femur and anatomical landmarks;

FIG. 3B is a representative 3D image of an enlarged view of the anterior view of a distal femur and anatomical landmarks;

FIG. 3C is a representative 3D image of a top view of the proximal tibia and anatomical landmarks;

FIG. 3D is a representative 3D image of an anterior view of a proximal tibia and anatomical landmarks;

FIG. 4A is a representative 3D image of an anterior view of a distal femur and calculated axes;

FIG. 4B is a representative 3D image of an enlarged view of the anterior view of a distal femur and calculated axes;

FIG. 4C is a representative 3D image of a top view of the proximal tibia and calculated axes;

FIG. 4D is a representative 3D image of an anterior view of a proximal tibia and calculated axes;

FIG. 5A is a bone jig for use in the surgical system of FIG. 1A;

FIG. 5B is a schematic showing the bone jig of FIG. 5A positioned and aligned with a projected hologram of an outer perimeter of the bone jig, on a femur;

FIG. 5C is a schematic showing the bony jig of FIG. 5A positioned and aligned with a projected hologram of an outer perimeter of the bone jig, on a tibia;

FIG. 6A is a schematic of the bone jig of FIG. 5A aligned with projected hologram, positioned on a femur;

FIG. 6B is a schematic of a femur and its mechanical axis;

FIG. 6C is a schematic of a femur and tibia in multiple positions during a method of determining mechanical axes;

FIG. 7A is a schematic of the bone jig of FIG. 5A positioned and aligned with a projected hologram of an outer perimeter of the bone jig, on a femur relative to the mechanical axis;

FIG. 7B is a schematic of the bone jig of FIG. 5A positioned on a tibia;

FIG. 7C is a schematic showing a femoral cutting jig aligned with a projected hologram of the femoral cutting jig, positioned on a femur;

FIG. 8A is an illustration of an example of a tibial implant;

FIG. 8B is an illustration of an example of a femoral implant;

FIG. 8C is an illustration of an example of an implant dispensing machine;

FIG. 9 is a diagram of an example of a surgical system that includes a handheld 3D scanner in accordance with the present disclosure; and

FIG. 10 is an example of a process for modifying a 3D model of a region of anatomical interest to include a fiducial in accordance with the present disclosure.

### DETAILED DESCRIPTION

Aspects of the present disclosure include surgical systems that provide a cost-effective, accurate, and efficient system for performing surgical procedures.

In one aspect of the disclosure, a surgical system utilizes an intra-operative laser, white light or blue light 3D scanner. This 3D scanner is used to determine anatomical landmarks and calculate surgical positions based on such anatomical landmarks. Utilizing well-defined focused light, e.g., laser light lines, onto a bony and/or a cartilage surface, the 3D scanner can be used to generate a complete or partial scan of the surgical surface, which can then be superimposed on pre-operative images to instantly register the bone. Such instant registration can be based on pre-operative imaging such as computerized tomography, magnetic resonance imaging, or plane radiographs of the limb or organ. In another aspect, the instant registration can be achieved with machine learning algorithms incorporating artificial intelligence technology.

In another aspect of the disclosure, a surgical system is provided that is useful in performing orthopedic procedures in the absence of trackers. In another aspect of the disclosure, a surgical system is provided that is useful in sizing orthopedic implants in the absence of an implant representative. In another aspect of the disclosure, an artificial intelligence system is used that utilizes machine learning to provide improvements in surgical efficiency. In another aspect of the disclosure, a surgical software system may be used to recognize and track implants, instruments or the like. In another aspect of the disclosure, a specific instrument can be used for calibration and aid in navigation or robotic assistance without trackers.

The present disclosure includes surgical systems that include one or more intra-operative 3D scanners. Although the surgical system is illustrated and described in the context of being useful for orthopedic surgical procedures, the present disclosure can be useful in other instances. Accordingly, the present disclosure is not intended to be limited to the examples and embodiments described herein.

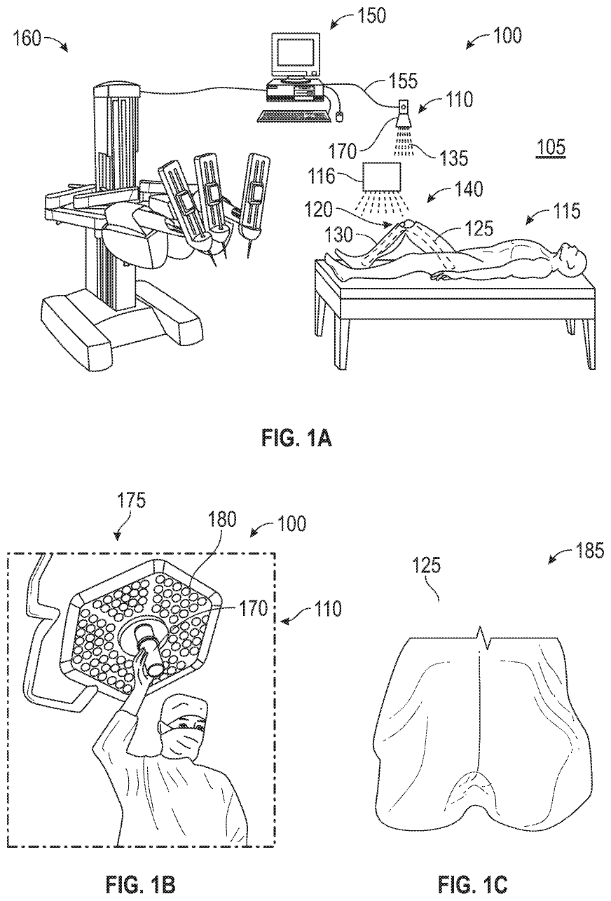

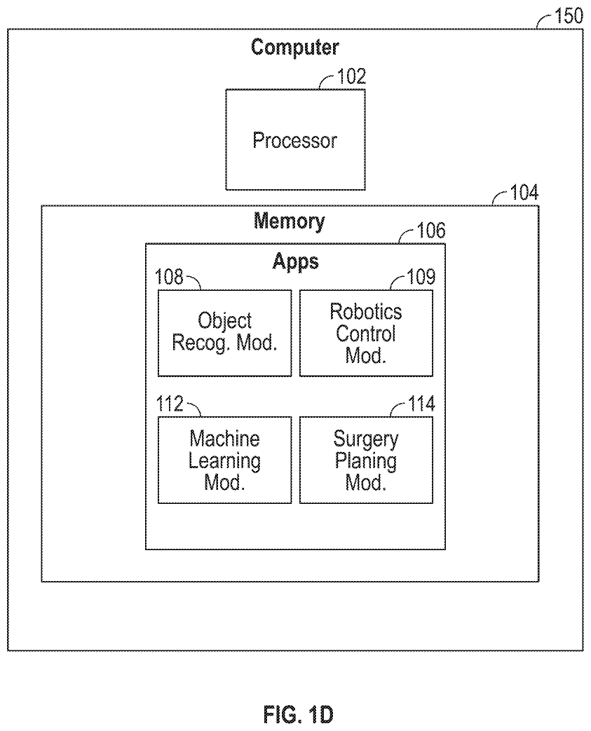

FIG. 1A shows a surgical system 100, which can be used to perform a computer-assisted surgery utilizing an intra-operative 3D scanner 110. The surgical system 100 of FIG. 1A is shown in use in an operating room 105 and includes a 3D scanner 110 capable of producing an intra-operative 3D scan of a body part of interest. In the context of FIG. 1A, a patient 115 is undergoing a knee replacement operation. The soft tissue around the knee 120 has been incised to expose the femur 125 and the tibia 130.

The 3D scanner 110 projects a light or other wave 135 onto the region of anatomical interest 140 and monitors the reflection of the light 135 so as produce a 3D scan of the region of interest 140. The 3D scan is transmitted to a computer 150 by cable 155 or by wireless connection. The computer 150 processes and analyzes the 3D scan and controls or assists the surgical procedure based on the analysis, as described below. For example, the computer 150 may control or operate or provide information to an optional robotics unit 160. The robotics unit 160 may perform a computer-guided surgical procedure. Alternatively, the computer 150 may provide information to a surgeon and/or may provide information to the robotics unit 160 that will allow the robotics unit 160 to aid the surgeon during the procedure.

Referring to both FIGS. 1A and 1D, the computer 150 can be any device capable of receiving input, performing calculations based on the input, and producing output as a result of the calculations. The computer 150 may include a central processor 102 that is capable of interacting with a user via a keyboard, a graphical user interface, wireless communication, voice command, or any other manner. The computer 150 may be a personal computer, a laptop, a handheld device, a server, a network of servers, a cloud network, or the like. The user, such as a surgeon or surgeon's assistant, may interact with the computer 150 before, during, or after the surgical procedure. The computer 150 may include a memory 104 or may be otherwise communicatively coupled to a memory that contains various software applications 106 for performing calculations, and executing algorithms, routines, and/or subroutines, for example, to process information and/or make determinations. For example, the computer 150 may include one or more software applications configured to analyze information (e.g. scan data) obtained from 3D scanner 110, generate a 3D model or 3D scan image, and analyze the 3D model. In one example, software applications 106 include an object recognition module 108 configured to recognize various objects or features in an image, such as the 3D scanned image. Facial recognition, fingerprint recognition, and iris recognition software systems are examples of object recognition technology. Each of these software systems make comparisons of anatomical features of an image with features in a database that is either stored in the computer 150 or is accessible by the computer by wired or wireless connection. The computer 150 may further include a robotics control module 109 for controlling and communicating with the robotics unit 160. The computer 150 may further include other optional modules, such as an artificial intelligence or also referred to herein as a machine learning module 112 that are configured to apply one or more machine learning algorithms to identify anatomical landmarks of interest, among other functions.

In examples, the 3D scanner 110 may be a laser, white light or blue light scanner. In various embodiments, the 3D scanner may be a device that generates 3D scan date that can be used to generate a virtual 3D model of a scanned object, which may include 3D scanner devices that perform surface height measurements of an object using coherence scanning interferometry with broadband light illumination. Commercially available 3D scanners that incorporate 3D scanning technology that may be used or modified for applications of the present disclosure include the AICON PrimeScan and the WLS400M from Hexagon Manufacturing Intelligence in Surrey, Great Britain; the Go!SCAN 3D from Advanced Measurements Labs in Tustin, California; the HandySCAN 3D TM from Creaform Inc. in Levis, Canada; and 3D scanners produced by E4D Technologies in Dallas, Texas.

As shown in FIG. 1B, in one example, the 3D scanner 110 is incorporated into handle 170 of medical light 175. Medical light also includes an array of lights 180 that are used to illuminate the operating room 105 as is known in the art. The 3D scanner 110 also includes one or more light emitting modules that may emit a laser, white light or blue light, which can be projected onto the patient 115 and the area of interest 140. 3D scanner 110 captures reflections of the light emitted by the scanner and which can be used to generate a 3D image using imaging software executed, e.g., by computer 150. As described herein, the term 3D model is often used interchangeably with the term 3D image. In the example shown in FIG. 1B, the 3D scanner 110 is mounted at the center portion of the medical light 175 at or near the handle 170 or in the peripheral aspect of the light 175 so that it may be easily manipulated and directed by a user, such as a surgeon or surgeon's assistant. The user directs the 3D scanner 110 at a region of anatomical interest 140, such as an exposed knee 120, and a 3D scan can be performed to generate a 3D image or model of the anatomy, such as the 3D image/model 185 shown in FIG. 1C.

FIG. 1C shows an example of a 3D image or 3D model generated from a 3D scan of an anterior view of the distal end of the femur 125. In one example, such 3D images or models are accurate up to less than 0.001 inches, with up to five million data points generated, e.g., in a few seconds, generating a nearly exact virtual model of the scanned object. The scan data generated by scanner 110 can be collected efficiently with minimal setups, and generated into a 3D image or model using, for example, one or more software modules executed by or accessible by computer 150 as described more below. System 100 may also include a hologram projector 116 for projecting a hologram of an object during surgery, which can be used for a variety of purposes, including projecting a proper position and orientation of a bone cutting jig in a surgical field.

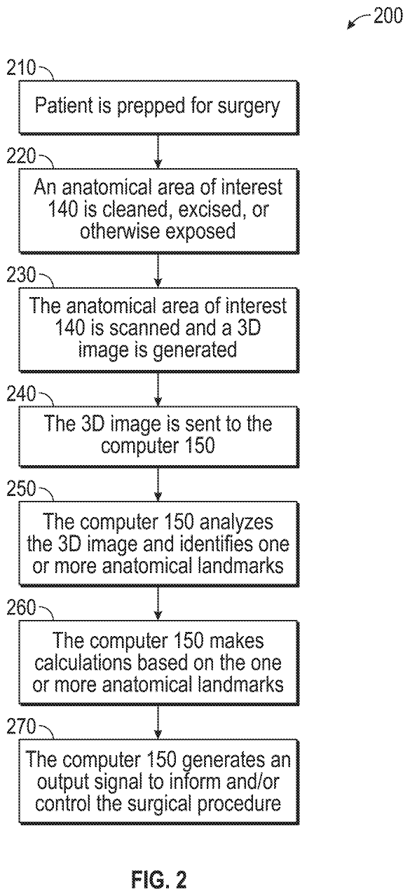

FIG. 2 illustrates an example of a surgical procedure 200 that may be performed using surgical systems of the present disclosure, e.g., surgical system 100. At step 210, a patient is prepped for surgery. At step 220, the anatomical area of interest 140 is cleaned, excised, or otherwise exposed so that it is visible from the point of view of the 3D scanner 110. Light 135 or other scanning medium is directed onto the anatomical area of interest 140 so that the 3D scanner 110 and/or computer 150 can generate, at step 230, a 3D image of the anatomical area of interest 140. The optical camera of the 3D scanner that is attached to the light handle is communicatively connected to the computer for transmitting images (e.g., scan data) for processing by the object recognition module 108. 3D scanner 110 and object recognition module 108 may be configured to constantly scan a field of view of the 3D scanner camera and automatically detect a scanned surface and anatomical landmarks located thereon. Object recognition module 108 can then automatically match or register the 3D scanner image to a preoperative image of the same anatomical area. If the 3D scanner includes separate processors and software for generating a 3D model or 3D image, then at step 240, the 3D image/model is sent to the computer 150 by cable connection 155, by wireless connection, or the like. At step 250, the computer 150 analyses the 3D image, for example, with object recognition module 108, and identifies one or more anatomical landmarks in the image.

The object recognition module 108 can be programmed or configured via a user interface to identify one or more particular anatomical landmarks. Once the one or more anatomical landmarks are identified, at step 260, surgery planning module 114 may be executed to perform calculations and/or make determinations based on the one or more identified anatomical landmarks. For example, surgery planning module 114 can determine the optimal location to make a cut or a drill a hole relative to the anatomical landmark. At step 270 the computer 150, e.g., with surgery planning module 114 can then generate an output signal related to the calculations or determinations. The output signal can be in any of various forms. For example, the output signal can be information that is delivered to the surgeon for the surgeon to consider during performance of the procedure. Alternatively or additionally, the output can be in the form of computer-assisted surgery signals or data, and the output can be used to guide pointers, instruments, and the like and/or can be in communication with a robotics module or a robotics unit 160. Alternatively or additionally, the output can be in the form of computer-aided design (CAD) files for use in computer assisted surgery, and the output can be used for providing visual aid on a monitor or via projecting, such as using hologram projector 116, onto the surgical field or on the skin or bony surface. The output can be used to guide pointers, instruments, robotic arms, and the like and/or can be in communication with a robotics module or robotics unit 160.

The surgical system 100 of the present disclosure is useful in a wide variety of surgical procedures where precise movements and/or placement of components relative to an anatomical landmark is important. For example, the surgical system 100 is particularly useful in orthopedic procedures where precise cuts and placement of devices is important for the success of the procedure. Joint replacement procedures, such as knee replacement and hip replacement procedures, are examples of such orthopedic procedures. The surgical system 100 is also useful in other surgical arenas, such as guidance of any cutting device. For example, the surgical system 100 can be used for fracture fixation with a plate or other fixation device. The 3D scan can help with superimposing an image onto intra-operative radiographs or fluoroscopic images. The surgical system 100 can also be useful in dental and maxillofacial surgical procedures; in spinal procedures especially when pedicle screws are to be placed by scanning the area and correlating with pre-operative and intra-operative MRI; hand, foot, and ankle procedures, shoulder replacement and fracture treatment procedures. In addition, the surgical system 100 can be useful in general surgical procedures where an organ is scanned by endoscopy and/or laparoscopy, and images are used to guide surgical tools for accurate cut or suture placement and the like.

The surgical system 100 will now be described in the context of a knee replacement procedure. The present examples and the specifics involved are not intended to limit the scope or usefulness of the surgical system 100 but merely to demonstrate its applicability in a particular field. One of ordinary skill in the art will understand that this exemplified use can be modified and extended to other fields, such as any of those mentioned above.

An important factor for a successful knee replacement procedure is the appropriate alignment and placement of implants to reproduce the biomechanical properties of the joint. Determination of proper alignment includes positioning the femur and tibia at a defined angle, typically 90 degrees, to the mechanical axes of the femur and tibia and typically within 3 degrees of error. As such, a cause for a malposition of an implant can be a 3-degree deviation from the 90-degree positioning to the mechanical axis or inappropriate rotation of femoral and/or tibial components. Accordingly, in one example, surgical system 100 may be designed and configured to aid in making the cuts associated with and placement of an artificial knee joint so as to be within the 3 degrees of the desired 90-degree positioning of the implant relative to the mechanical axes of the femur and tibia.

Memory 104 may include information related to the knee joint and the instruments associated with knee joint replacement, with such information accessible by object recognition module 108 and surgery planning module 114.

For example, the computer 150 may execute object recognition module 108 and recognize a pre-defined bone jig configured for use in the procedure, as well the anatomy of the knee 120. After the surgical approach is performed and the knee exposed, the medical lights 175 equipped with a 3D scanner 110 like the one in FIG. 1B may be brought closer to the knee region 120, a 3D scan of the exposed bone can be performed, and a 3D image is generated. In one example, a plurality, e.g., two, 3D scanners 110 can be utilized. The plurality of 3D scanners 110 can be positioned at different locations around knee region 120 so that they generate a corresponding plurality of different simultaneous views of the exposed surgical area. The 3D image can then be delivered to the computer 150 by Wi-Fi technology or the like, or scan data generated by the scanners can be transmitted to the computer to generate a 3D image or model. Object recognition module 108 can be configured to recognize and detect different surface textures and colors and can distinguish between bone, cartilage, instruments, and soft tissue. The 3D image can be analyzed by object recognition module 108 to identify pre-determined anatomical landmarks.

For the knee replacement surgery, the object recognition module 108 may be configured to identify certain predetermined anatomical landmarks. For example, one or more of bony landmarks, surfaces, limb axes, and dimensions can be identified and defined or recorded by the object recognition module 108 and stored in the memory 104.

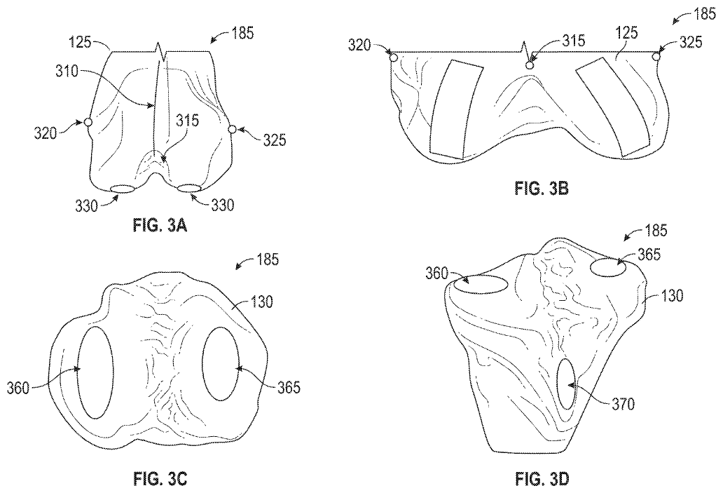

FIGS. 3A through 3D illustrate examples of the anatomical landmarks that object recognition module 108 may be configured to identify and locate. FIG. 3A is a representative 3D image 185 of an anterior view of the distal femur 125 generated from a 3D scan of the distal femur. FIG. 3B is an enlarged anterior view of a portion of the distal femur 3D image 185. FIG. 3C is a representative 3D image 185 of a top view of the proximal tibia 130. FIG. 3D is a representative 3D image 185 of an anterior view of the proximal tibia 130. On the femur 125, object recognition module 108 may be configured to identify one or more of the trochlea groove 310, the trochlea notch 315, the medial epicondyle 320, the lateral epicondyle 325, and the distal femur articulating surface 330. On the tibia 130, object recognition module 108 may be configured to identify one or more of the medial tibial plateau 360, the lateral tibial plateau 365, and the tibial tubercle 370. In another example, object recognition module 108 may be configured to identify one or more predetermined bone-cartilage junctions as one of the anatomical landmarks. In one example, the computer 150 may be used to identify and locate all of the above landmarks on the femur 125 and the tibia 130.

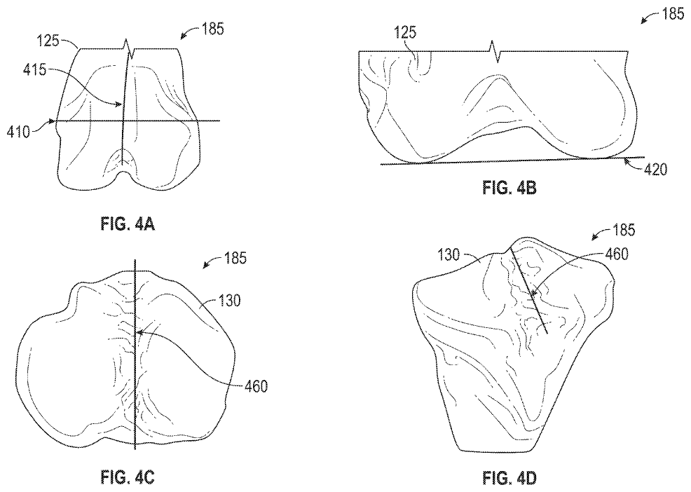

After identifying and locating the anatomical landmarks, surgery planning module 114 may be executed to perform calculations based on the landmarks. For example, FIGS. 4A through 4D show the representative 3D images 185 from FIGS. 3A through 3D respectively, and also illustrate pre-established axes calculated by surgery planning module 114 for implant positioning. On the distal femur 125, surgery planning module 114 may calculate the transepicondylar axis (TEA) 410, the patetllofemoral axis (PFA) 415, and the posterior condylar axis (PCA) 420. On the proximal tibia 130, surgery planning module 114 may calculate the tibial rotation axis (TRA) 460.

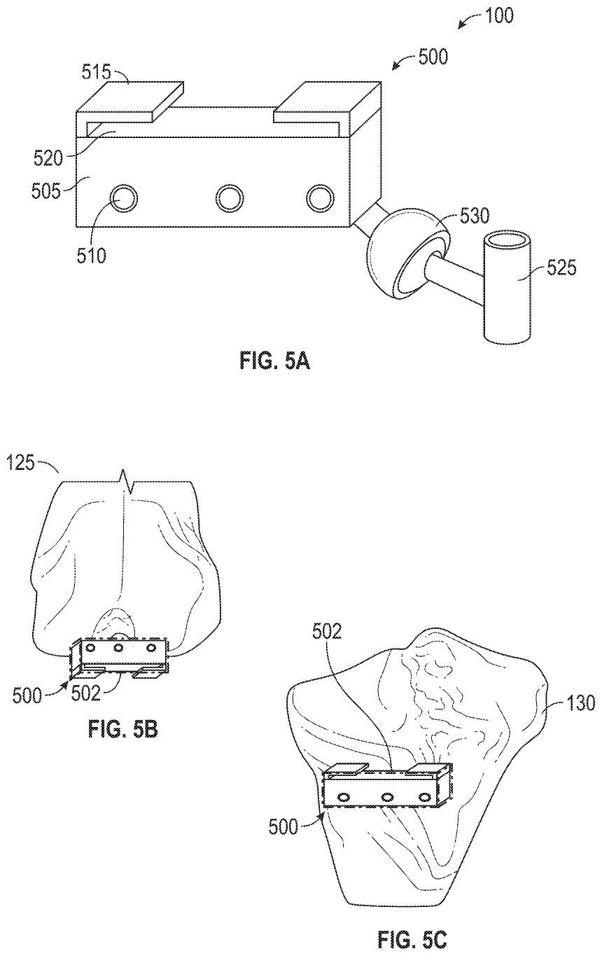

In one example, surgical system 100 further includes a bone jig 500 (FIG. 5A). In the illustrated example, bone jig 500 is a bone cutting guide and memory 104 may contain one or more dimensions of the jig. The bone jig has a body 505 with pin holes 510 for fixation to the bone. A saw blade protector 515 helps define a guide slot for a saw blade. Bone jig 500 also includes an initial fixation pin hole 525 coupled to body 505 by a hinged connection 530, which as described below, can be used for fine adjustments of the bone jig 500 prior to fixation of the jig t bone with pin holes 510. The bone jig 500 is relatively small and is user friendly. The bone jig 500 is positioned over the femur 125 in FIG. 5B and over the tibia 130 in FIG. 5C at precisely determined positions as will be described. FIGS. 5B and 5C also illustrate a hologram projection 502 projected onto the bony surface from hologram projector 116 and show the jib aligned with the projection. In the illustrated example, the projection is a projection of a portion of an outer perimeter of the bone jig. In other examples, other types of projections may be used, such as the projection of one or more points. The jig position can, therefore, be projected onto the bony surface, so that the surgeon can position the jig with the projected hologram. As will be appreciated, hologram projector 116 can also be configured to project other holograms, for example, one or more targets or a portion of an outer perimeter of other jigs. In other example, rather than aligning a bone cutting jig, such as jig 500, with a hologram, such as hologram 502, a hologram projection may be directly used as an augmented reality cutting guide and a surgeon may use a surgical instrument, such as a saw in a plane of cut that is projected by the hologram.

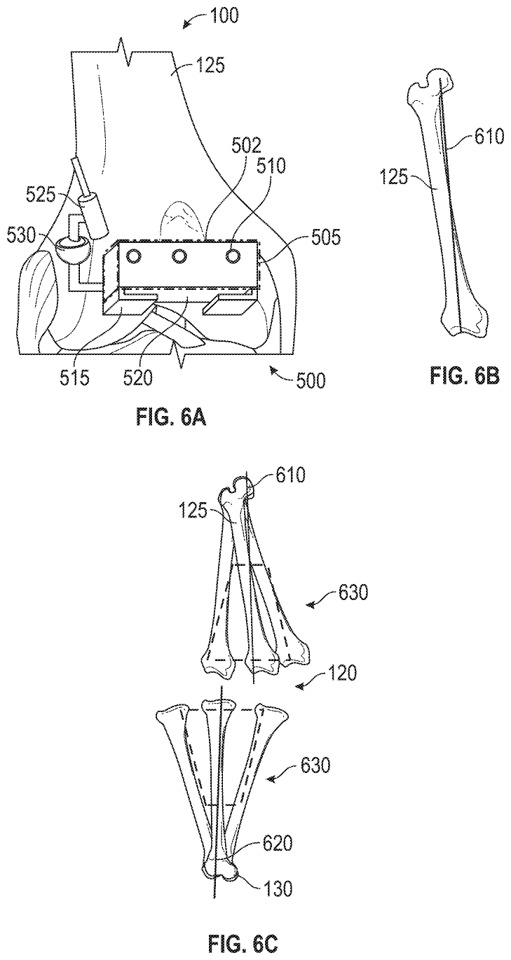

Since the bone jig 500 has exact pre-determined dimensions, it can also be used by the computer 150, e.g., surgery planning module 114, to calibrate images (for example, in cases where there are no pre-operative images) of the bone jig captured by 3D scanner 110. The bone jig 500 parameters and dimensions are loaded into the computer 150 and stored in Memory 104 prior to surgery. Then, during surgery, object recognition module 108 can be configured to detect the unique shape and dimensions of bone jig 500 and, in some examples, since the dimensions are already defined, the dimensions can be used to calibrate the image of the scanned bone adjacent to the bone jig. With the jig 500 roughly positioned in a region of interest, a pin can be inserted through the initial fixation pin hole 525 and the bone jig 500 is placed over the bone and the bone jig 500 can be provisionally fixed by this pin to the bone (as shown in FIG. 6A). The computer 150 recognizes the bone jig 500, the 3D image of the bone, and the calibrated bone.

The mechanical axis 610 of the femur and the mechanical axis 620 of the tibia are determined as shown in FIGS. 6A through 6C. With jig 500 provisionally fixed to the bone, the knee can be placed in different positions, moved around in a triangle 630 until the mechanical axis of the bone is identified from this triangular positioning. This is done based on the shape of the cutting jig, distance and position as referenced to the optical camera of 3D scanner 110, e.g., on the light handle 170. Bone jig position data can be determined from the image data captured by the camera of the 3D scanner 110 with, e.g., surgery planning module 114, and stored in Memory 104. Surgery planning module 114 may also be configured to calculate the femur mechanical axis from the bone jib position data. The rotational axis of the femur can also be calculated based on transepicondylar axis or gap balancing principles, which are previously described and well-known in the art. Since the mechanical axis of the femur 125 goes through the femoral head, by rotating the distal aspect of the femur in various positions, the position and orientation of the bone jig 500 and the bony surface can be determined from images of the jig and bone surface captured by the camera of the 3D scanner 110, and the computer 150, with, e.g., surgery planning module 114, can generate a model that defines the femur mechanical axis. This axis is used for cutting the distal femur 125. Similarly, the tibial mechanical axis is defined based on the change in position and orientation of the jig 500 fixed to the proximal tibia, determined from analysis of images of the jig captured by the optical camera of the 3D scanner while the tibia is rotated around the ankle axis.

These axes are important for proper implant positioning as the bony cuts and thus the implants are desirably placed 90 degrees to the mechanical axes 610, 620. After the mechanical axis of the femur 610 and the mechanical axis of the tibia 620 are defined, surgery planning module 114 can determine the proper positions of the bone jig 500 over the bony surface. Surgery planning module 114 can also be configured to generate an image of the proper position of the jig on the bone that can be overlaid with a live image of the bone surface displayed on a monitor of computer 150. The surgeon can adjust the position and orientation of the jig on the bone surface while watching the monitor until the live image of the jig is aligned with the properly positioned image generated by the surgery planning module 114. In some examples, hologram projector 116 may also be used to project a hologram of a properly positioned jig on the bone surface, which the surgeon can use to align jig 500. The calculated jig position and orientation can be modified based on the surgeon's preferences and techniques and can also be modified pre-and intra-operatively to accommodate different bony resection methods (measured resection, gap balancing and kinematic or a combination thereof). The jig position and orientation can also be pre-defined based on the surgeon's preferences and techniques.

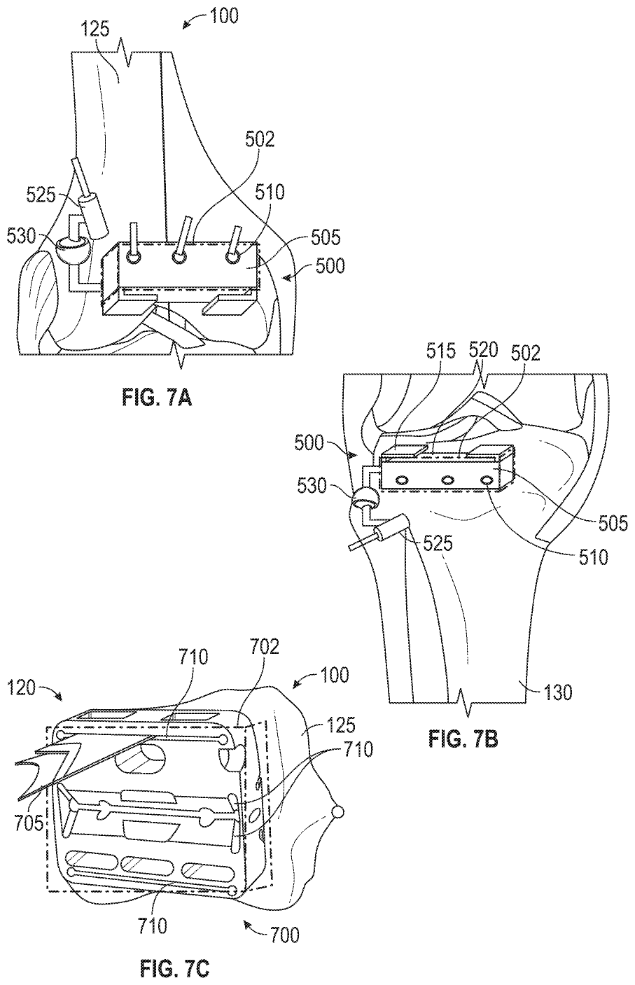

As shown in FIGS. 7A through 7C, surgery planning module 114 can be executed to calculate the optimum position of the bone jig 500 for restoration of bony cut in three planes: Medial-lateral, anterior-posterior, and superior-inferior planes. For the femoral cut, as shown in FIG. 7A, surgery planning module 114 may determine the perpendicular axis to the mechanical axis of the femur and calculate the position of the jig to obtain appropriate depth of bony resection, as well as alignment in three planes. The bone jig 500 can then be fixed to the femur 125 with multiple pins using the methods described above, e.g., when the bone jig 500 is superimposed accurately on a projected hologram from hologram projector 116 and the surgeon has achieved all the qualifying criteria for the bony cut (which are based on principles of knee arthroplasty), including depth of the cut and the location of the cut in the three planes. Alternatively, the surgeon can watch a live image of the knee region that includes a computer-generated cutting jig in the proper position and orientation.

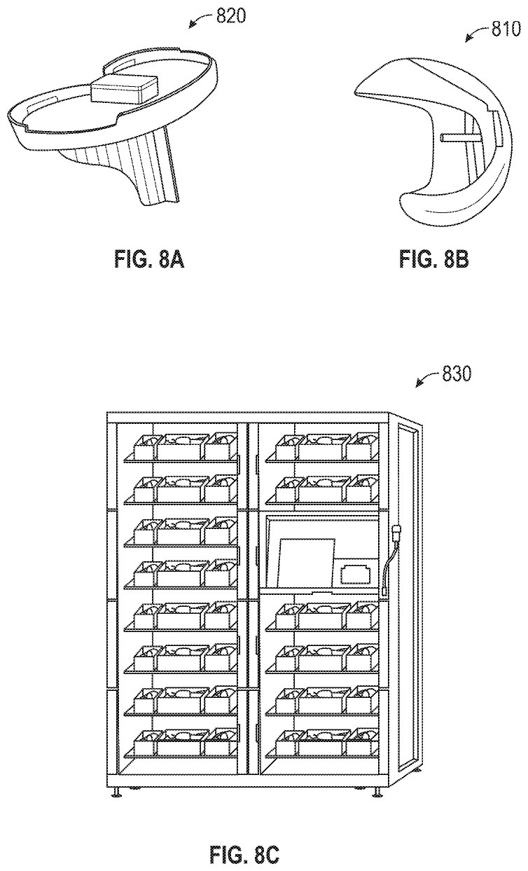

FIG. 7B shows the jig positioned for the tibial cuts. After the proximal tibia and distal femoral cuts, the bone cutting jig(s) 500 are removed but the initial pins can be left in place. Then a spacer block (not illustrated) can be placed in the knee 120 in extension. The soft tissue balance of knee is assessed in extension with varus/valgus forces manually applied. Scanner 110 can continuously monitor the movement of the pins during the varus/valgus test and the change in position of the pins can be calculated by the computer 150, e.g., surgery planning module 114, which can be used to determine the medial and lateral opening in extension. This opening is usually 2-4 mm. If the extension gap is not balanced, the surgeon can perform various methods known in the art to achieve a balanced extension gap. Then the knee is placed in 90 degrees of flexion and distracted by manual means or use of lamina spreaders. The femoral 4-in-1 cutting jig 700, which is typically provided by the implant manufacturing company and specific to the size of the implant is placed over the distal femoral cut. The rotational orientation of the femoral 4-in-1 cutting jig 700 can be determined based on anatomic landmarks identified by object recognition module 108 and re-creation of a rectangular flexion gap. The computer 150 has the ability to identify this instrument and communicate with the surgeon as displayed on the monitor or hologram projector, as what the appropriate position should be to achieve a balanced flexion gap. Femoral sizing can be performed by surgery planning module 114 based on implant dimensions stored in Memory 104 for femoral implants 810, such as the one shown in FIG. 8B, bony landmarks that were identified previously and stored in memory and the calculated flexion gap. In one example, the flexion gap is achieved by “parallel to the tibial cut” technique, distracting the femur in 90 degrees of flexion. Femoral sizing and rotation can be adjusted intra-operatively if the surgeon needs to up or downsize the implant to achieve accurate flexion and extension gaps. The tibial implant 820 (FIG. 8A) is then similarly sized. After cutting the anterior, posterior, chamfers using a bone saw 705 inserted into the cutting slots 710 in the 4-in-1 cutting jig 700 as shown in FIG. 7C (the alignment of the 4-in-1 cutting jig 700 being guided by hologram 702), trial implants are used to assess the gaps and alignment prior to opening the final implants. Surgery planning module 114 can determine the correct size of the trial implants and communicate with an implant dispensing machine 830, as shown in FIG. 8C, to open the appropriate door for the proper implant and reduce errors. Computer 150 can also send an email for replenishment and a bill after the implant is used.

The implant dispensing machine 830 can be operated by, e.g., nurses in an operating room and can eliminate the need to have an implant representative present in the operating room for routine cases. The ability to integrate the surgical system 100 and a facility's billing department can also be beneficial.

In the illustrated example, the implant dispensing machine 830 includes actual implants provided by one or more manufacturing companies and the machine is replenished by the corresponding companies. Implant dispensing machine 830 can also store disposable items such as instruments and jigs.

Although described in this example in the context of a knee replacement operation, the surgical system 100 can be similarly used in hip replacement and shoulder replacement procedures, as well as other procedures mentioned above.

In hip replacement procedures, the surgical system 100 can calculate functional anteversion and abduction angles in adjusted zone. The computer 150 can feature broach recognition, femoral anteversion and depth of broach based on pin location. The surgical system 100 allows for only one reamer to be necessary during pelvic preparation, provides depth of ream, anteversion and abduction angles for final cup positioning. Lastly, the surgical system 100 can capture the final data and store it on the patient's file and generate operative reports for better documentation.

In one example, system 100 can be used to perform a surgery without conventional instruments, traditional manual alignment jigs, pre-operative CT scans, trays, or sterilization of multiple trays during surgery, which can significantly increase OR efficiencies and thus simplify knee and hip surgeries. In other examples, system 100 can be used in combination with one or more of the above to improve the accuracy and efficiency of a surgery.

The surgical system 100 of the present disclosure provides an accurate, affordable, easy to use open-platform navigation system for reproducible and correctly-performed hip and knee replacement or other surgical procedures. The surgical system 100 can be used to eliminate one or more of current traditional instruments, can make a surgery less complicated, eliminate trays, sterilization processes and reduce costs while improving outcomes. The surgical system 100 can also be used to improve the surgical flow and make a surgery faster with less errors. In addition, implant dispensing machines 830 can reduce errors in implant utilization by eliminating human errors, improve billing processes and provide for auto-replenishment of implants.

The surgical system 100 uses 3D intra-operative laser, white, or blue light scanner(s), which may be attached to a medical light above a patient. In one example, the system obviates the need for trackers, which are typically used in prior art computer-aided navigation to aid with registration as a fixed point on the bone.

Aspects of the present disclosure also include, in one example, a method of performing a surgical procedure, comprising: scanning, with a 3D scanner, a region of anatomical interest; generating, with a processor, from data generated by the 3D scanner during the scanning step, a 3D image; identifying, with the processor, in the 3D image, one or more anatomical landmarks; calculating, with the processor, according to the identified anatomical landmarks, a plurality of surgical positions; and generating, with the processor, guidance information, according to the surgical positions, for guiding a surgical procedure.

Aspects of the present disclosure also include a computing device, comprising: a 3D scanner and; a processor configured to: receive, from the 3D scanner, scan data from a scan of a region of anatomical interest; generate, from the scan data, a 3D image; identify, in the 3D image, one or more anatomical landmarks; calculate, according to the identified anatomical landmarks, a plurality of surgical positions; and generate guidance information, according to the surgical positions, for guiding a surgical procedure.

Aspects of the present disclosure also include: a surgical system useful in performing orthopedic procedures in the absence of trackers; a surgical system utilizing an intra-operative laser 3D scanner, wherein the 3D laser scanner is used to determine anatomical landmarks and/or calculates surgical positions based on the anatomical landmarks; “instant registration” that can be based on pre-operative imaging such as computerized tomography, magnetic resonance imaging, or plane radiographs of the limb or organ; instant registration based on machine learning and artificial intelligence; an object recognition module that includes code, algorithms and/or routines, that allow for identification of the actual surfaced area based on the 3D scan; software that recognizes the scanned bone and determines a proper placement of a pin for which all calculations are based on, for example one pin is placed on the femur and one on the tibia during a knee replacement; software that can recognize the distance change between two pins, which is used for soft-tissue assessment; software that can recognize and track the implants, instruments or the like; an object recognition module can also recognize the cutting jigs/instruments; a computer screen that can show the plane of the bony cut so the surgeon can align the jig and the cutting planes; an implant dispensing machine that can store multiple sizes of an implant; and a computer that can identify the size of implant trials and communicate with an implant dispensing machine to open an appropriate door for a specified implant and reduce errors.

As noted to some degree above, various implementations consistent with this disclosure provide systems, methods, devices, and computer products for new intra-operative registration techniques that may be used with image-based CAS systems, such as image-based surgical navigation systems, and the like, e.g., for orthopedic procedures. Such implementations involve the superimposition, alignment, or registration of a virtual 3D model of an area of anatomical interest that is built from scan data gathered during surgery (intra-operatively) using a 3D scanner device with pre-operative images of the area of anatomical interest so as to accurately identify and track the position of a fiducial that is attached in the area of anatomical interest by the surgeon during surgery. Registration is required to orient the CAS system's coordinate system to the real-life surgical coordinate system and thereby enable accurate real-time tracking of the patient anatomy using an image-based CAS navigation system. The accuracy of the registration of the anatomic landmarks (e.g., bone features) directly affects the overall accuracy of the CAS navigation system. In many embodiments, the CAS navigation system may an optical tracking system that uses cameras and optical trackers affixed to bones etc., while in other embodiments, the CAS navigation system may be a radar tracking system that uses radar transceivers and radar-beacon trackers affixed to bones, etc. (e.g., as described in U.S. Pat. No. 11,896,319 and its family members).

In various implementations, the present disclosure is directed to systems, methods, devices, and computer products for CAS that intra-operatively utilize a handheld scanner (e.g., a 3D laser scanner) to captures and create and intra-operative 3D model of medical surfaces, (e.g., bone and cartilage surfaces, the surfaces of a surgeon-attached fiducial, etc.) and register the intra-operative 3D model with the pre-operative 3D images/models, (such as MRI, CT, or similar pre-operative 3D images/models), utilized by a CAS navigation system such that the fiducial surfaces become part of the 3D model used by the CAS navigation system, and such that the CAS navigation system can track the location of the fiducial and the medical object (e.g., bone) to which the fiducial is attached.

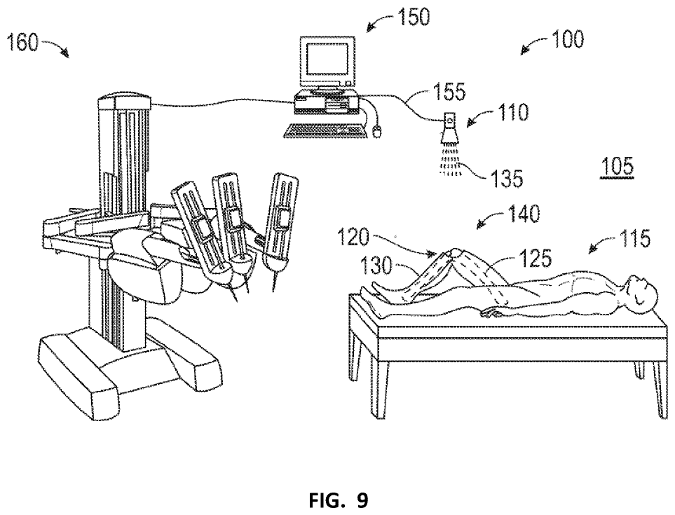

FIG. 9 is a diagram of an example of a surgical system 100, such as a CAS system, that includes a handheld 3D scanner 110 in accordance with the present disclosure. The system 100 can be used to perform a computer-assisted surgery utilizing the intra-operative 3D scanner 110. The surgical system 100 of FIG. 1 is shown in use in an operating room 105 and the handheld 3D scanner 110 is capable of intra-operatively producing scan data for an intra-operative 3D model of a region of anatomic interest 140, such as a body part of interest. In the context of FIG. 9, a patient 115 is undergoing a knee replacement operation and the leg of the patient 115 in the knee area is the relevant region of anatomical interest 140. The soft tissue around the knee 120 has been incised to expose the femur 125 and the tibia 130. For clarity in the drawing, the surgeon (or other user) that is holding the scanner 110 in their hand is not shown in FIG. 1. It should be noted, nonetheless, that the scanner 110 may be easily moved, positioned, and pointed by the user (not shown) that is holding it. Thus, the user can move the 3D scanner 110 all around the exposed femur 125 and tibia 130 so as to gather enough scan data to create a good intra-operative 3D model of the tibio-femoral joint, including the relevant portions of the femur 125 and the tibia 130. It should also be noted that the surgeon can attach fiducials, such as are described below, to the femur 125 and/or to the tibia 130 before scanning, so that the fiducials are included in the intra-operative 3D model of the femur 125 and the tibia 130.

The 3D scanner 110 projects a light (e.g., laser) 135 onto the region of anatomical interest 140 that the user points it at and monitors, captures, and/or processes the reflection of the light 135 so as produce scan data, which is used to form a 3D model of the region of interest 140. In various embodiments, the 3D scan data is transmitted to a computer 150. In one embodiment, the 3D scanner 110 transmits the scan data via a wire or cable 155 as shown in the example of FIG. 1, and the wire 155 should be long enough to easily reach all around the region of interest 140. In other embodiments, the transmission may be via a wireless connection, (e.g., Bluetooth, or the like). In various embodiments, the computer 150 processes the 3D scan data to produce an intra-operative 3D model, which, as noted, includes a representation of any fiducial(s) attached to the bones by the surgeon.

In various implementations, the surgeon or other user may position the handheld 3D scanner 110 to start the scanning process from a known point, feature, or landmark on a bone that is used as an initial fixed reference basis. As noted, the surgeon can affix a fiducial to a bone prior to scanning and in some implementations the fiducial may be used as the scan starting point.

In some general implementations, a fiducial may be an object with a known topology that is easily distinguishable from bone surfaces, skin surfaces, etc. in a scanned 3D model, such as a pyramidal topology. In some embodiments, the fiducial may be a plate shaped object, which may be metal or polymer. Some plate fiducials may have an easily identifiable top surface topography, so as to make them easy to distinguish from bone and cartilage surfaces, and plate fiducials may also be designed for a tracking device (a.k.a. a navigation marker) to attach to them. In other embodiments, the fiducial may be a non-plate-shaped anchoring device that is configured such that an additional device(s) may be easily and securely attached to it, such as a tracker device. Examples include an optical tracker device used with a CAS optical navigation system 160 or a radar beacon tracker device used with a CAS RF-based navigation system 160. In some instances, an extension arm may attach to the anchor fiducial, and the tracker device may in turn attach to the extension arm, such that the arm raises the tracker device above the patient's soft tissue. One example of an anchor fiducial is a metal anchor that mounts to a bone using teeth and a screw and that includes attachment features for tracking devices, etc. to mount on it, as described (e.g., FIG. 20A) in U.S. patent application Ser. No. 18/124,554, filed on 21 Mar. 2023, which is hereby incorporated by reference in its entirety. In some other embodiments, the fiducial may be or include a complete tracker device, such as an optical tracker device featuring four reflector spheres arranged in a specific configuration.

In some implementations, the fiducial may be secured to a bone at a specific location, which may be determined in advance. In some such implementations, the predetermined location may be identified using a pre-operative 3D model or pre-operative image(s), such as a computer tomography image, an ultrasound image, a magnetic resonance image, or the like. In some embodiments, a bone-affixed fiducial (e.g., a metal plate) can include a bar code (e.g., QR code) or the like that includes, or provides a reference to, pre-determined surgery-assisting data or information, such as: surgical steps, a surgeon's preferences (e.g., preferences for cuts, angles, equipment settings, etc.), patient data, etc. In various implementations, this surgery-assisting data may be detected and relayed to the computer 150 by the scanner 110, or by a separate bar code scanner/reader (not shown). In other implementations, the surgeon may attach a fiducial to a bone at a location that the surgeon determines intra-operatively—i.e., without using a predetermined location.

In either implementation, the 3D scanner 110 may be used to scan the fiducial after the surgeon affixes it to the bone, and the computer system 150 may analyze the intra-operative 3D model to intra-operatively determine and/or record the location of the affixed fiducial. The computer system 150 may also provide the location of the affixed fiducial to a CAS navigation system 160, for example, by recording or registering the fiducial's location in the 3d model used by the CAS navigation system 160.

As shown, the surgical system 100 includes a CAS unit 160, such as a CAS navigation (e.g., tracking) system, a surgical robot unit, or the like, which may be communicatively connected to the computer 150, as shown. In some typical embodiments, the CAS unit 160 includes and/or uses a pre-operative 3D model that was created from pre-operative imaging data, including non-optical imaging data, such as MRI scan data, CT scan data, X-ray imaging data, or other imaging techniques from which a 3D model of anatomy can be constructed. In various embodiments, a pre-operative 3D model created from pre-operative MRI scan data may be preferred, especially for orthopedic surgery, because the MRI model may better represent the cartilage in comparison to a CT-based model. The CAS unit 160 uses the pre-operative 3D model in order to perform its functions, such as real-time tracking of optical tracker devices, or the like, that are affixed to the bones (e.g., using an anchor fiducial). In various implementations, the pre-operative 3D model, or a copy thereof, may be stored by the computer 150. It also may be stored by the CAS unit 160. In various embodiments, the pre-operative 3D model and/or the intra-operative 3D model may be produced using commercial software, such as the MIMICS software by the Materialise company of Leuven, Belgium.

In various implementations, the computer 150 may register the intra-operative 3D model associated with the 3D scanner 110 with the pre-operative 3D model associated with (e.g., used by) the CAS unit 160. In general, this may be done by scaling and turning the intra-operative 3D model until its virtual surface nearly matches the virtual surface and orientation of the pre-operative 3D model. In some implementations, an iterative closest point (ICP) algorithm may be used to align, match or register the scan-based intra-operative 3D model with the MRI-based pre-operative 3D model. The ICP algorithm identifies the rigid transformation that best fits a cloud of points with a model. The algorithm uses a least-squares method to minimize the sum of the squared differences between both sets of points. In some embodiments, the algorithm may be preset to use 85% of the point cloud for matching to allow for surface variations and outliers, including variations caused by the fiducials that are included in the intra-operative 3D model but not in the pre-operative 3D model. Other percentages may also be used. In some embodiments, the computer 150 may determine the accuracy of the registration fit based on the Root Mean Square (RMS) difference between the two surfaces.

In various implementations, the registration function may perform a spatial alignment of the coordinate frame of the CAS navigation system 160 (e.g., the pre-operative 3D model) with the coordinate frame of the real-world patient 115 as represented by the scanned-in intra-operative 3D model. The spatial alignment process may utilize anatomic objects (e.g., landmarks on bones) to aid in, and increase the accuracy of, the alignment.

In some embodiments, in aligning the two models, the computer 150 may create a combined 3D model, or the like, that includes features from both models, which features may include the fiducial(s) represented in the intra-operative 3D model. In such embodiments, the computer 150, in effect, adds the 3D representation(s) of the fiducial(s) to the pre-operative 3D model, based on the location of the fiducial(s) as recorded by the 3D scanner 110. In some embodiments, this combining may be done, for example, by overlaying or superimposing the intra-operative 3D model and the pre-operative 3D model. A CAS navigation system 160 may use the combined 3D model with the fiducial representation(s) to track the location of the fiducial(s), and thus also track the bone(s) to which the fiducial(s) are attached. This is in contrast to the pre-operative 3D model which had no information about the location of the fiducial(s) in relation to the bones (or anything else), such that a CAS navigation system 160 would not be able to identify or track the fiducial(s) or the bones using just the pre-operative 3D model.

Expressed another way, the herein described techniques associated with using the 3D scanner 110 to create an intra-operative 3D model after a fiducial is attached to a bone provides a novel, fast, low-error, technical solution to the problem of how to add the location of a fiducial to an existing pre-operative 3D model that is used by a CAS navigation system 160 or the like.

In operation, the CAS navigation system 160, e.g. an optical navigation system 160, may locate and track the tracking device (e.g., a navigation marker featuring four reflector spheres arranged in a specific configuration) in its detection space in the operating room 105. And using the known spatial relationship between the tracker device and the anchor fiducial to which it is attached, as well as the known position of the anchor fiducial on a bone as represented in the pre-operative 3D model as modified by the intra-operative 3D model which includes the anchor fiducial, the optical navigation system 160 can determine and display the current position of the bone to which the anchor fiducial is attached. This is rather similar to the tracking of the bone jig 500 described above.

Additionally or alternatively, the computer 150 may control, assist, or provide directions or steps for surgical procedure(s), as described herein. For example, the computer 150 may control or operate or provide information to the CAS unit 160. In some embodiments that use a surgical robot unit 160, the surgical robot unit 160 may perform a computer-guided surgical procedure using, for example, the fiducial locations from the intra-operative 3D model. Additionally or alternatively, the computer 150 may provide information to a surgeon, such as a workflow, which may include multiple steps for a surgical procedure. Additionally or alternatively, the computer 150 may provide information to a CAS navigation unit 160 that presents displays (e.g., 3D model displays), suggestions, directions, etc., using, for example, the fiducial locations from the intra-operative 3D model, to aid the surgeon during an operation, such as a total knee arthroplasty (TKA), among others.

In various implementations, the computer 150 may be a device as described above with respect to FIGS. 1A and 1D. In various implementations, the handheld 3D scanner 110 may be a device as described above with respect to FIG. 1A. In some implementations, the 3D scanner 110 may obtain or utilize a series of snapshot images, and the computer 150 may analyze and stitch together the scan data representing the snapshot images of the scanned surfaces by overlapping the edges of each scanned surface image with each other, so as to form a more complete 3D model of the region of interest.

In some implementations, the intra-operative 3D scanner 110 may be mounted and set to continuously scan the fiducial, and the system 100 may process the continuous scan data to track the fiducial in real time or near real time; e.g., to provide live tracking of the fiducial. In some implementations, the scanner 110 may calculate or measure the distance from the 3D scanner 110 to the region of interest (e.g., to a bone surface, landmark, etc.) during the course of an operation, e.g. at short intervals, and the computer system 150 may use the distance measurements for tracking the area of interest. Additionally or alternatively, the distance measurements may be provided to a CAS navigation system 160, which may perform the tracking function. In such variants, the CAS navigation system 160 may use the distance measurements from the 3D scanner 110 to supplement its own independent distance measurements. In other similar variants, the CAS navigation system 160 may use only the distance measurements from the scanner 110 to perform its tracking function. In implementations that are configured for scanner tracking, the scanner 110 may be mounted at a fixed location known to the system 100, for example, it may be placed in a wall-mounted cradle or stand-mounted cradle, while the surgeon performs the surgery. In some embodiments, the scanner 110 may be configured to ignore erroneous data and images related to temporary obstructions, such as the surgeon moving their hand between the scanner 110 and the region of interest 140 that is being tracked.

In some embodiments, a pre-determined workflow may be displayed or otherwise provided by the computer system 150 (e.g., via its graphical user interface screen) to indicate to the surgeon where to start the initial scan using the handheld scanner 110 and to indicate each step of the surface scan procedure, so as to produce a satisfactory intra-operative 3D model. For example, the computer 150 may display steps indicating which separate anatomical features or landmarks to scan and the order in which to scan them. In such embodiments, the surgeon may position and then activate the handheld 3D scanner 110 according to the steps provided by the computer system 150. In such embodiments, the system 150 may identify or tag different image(s) as being associated with specific bony landmarks, based at least partially on which step of the pre-determined workflow has gathered or produced each set of scanned images.

In some implementations, the light output 135 of the 3D scanner 110 (e.g., the laser light) may be set, modified, or changed by the user, e.g., based on the anatomical area of interest 140 that the user is going to scan, the desired amount of detail or area coverage, and/or e.g., based on instructions from the pre-determined workflow provided by the computer system 150. The scanner 110 may employ lenses, apertures, multiple emitters, or other optical devices or techniques to change its light output 135. For example, the light beam or laser beam 135 may be widened so as to scan a larger area in one step (e.g., in one image) rather than taking multiple, smaller, snapshot images to cover the same area. For another similar example, additional light emitters (e.g., lasers) of the scanner 110 may be activated to emit more beams 135 and scan a larger area in one step (e.g., in one image).

In various implementations, the computer system 150 may analyze the scan data gathered by the handheld 3D scanner 110 and/or the intra-operative 3D model using machine learning models and artificial intelligence, for example, to identify anatomical features or landmarks (e.g., bony landmarks). In some embodiments, the computer system 150 may employ predictive modeling to perform such identification. The identifications may be used to aid in the registration process and/or to identify the location of fiducials that are attached to the bone that includes the landmarks.

In various implementations, the computer system 150 may analyze the scan date and/or 3D models in order to differentiate between different types of tissue, for example, between bone tissue and cartilage tissue. In some implementations, the computer system 150 may analyze the color and/or texture of a bone surface image in order to identify features, such as anatomical landmark features or the like.

Other variants may be used for imageless CAS navigation systems 160. In imageless systems, there are no prior advanced images and thus no pre-operative 3D model, and the surgeon is required to either “paint” the surfaces of the region of interest 140 with a pointer probe to obtain the data needed by the CAS navigation system to build an fairly accurate 3D model, including the joint surfaces, or manually choose the most important anatomical landmarks in the region of interest 140 using the pointer probe, which typically results in a less accurate 3D model. In either case, the manual use of a pointer probe to create a 3D model introduces user error and variability when recording bone morphology for the 3D model. Using a 3D laser scanner as disclosed herein is an easy and accurate method of acquiring bone morphology to create the 3D model and it requires much less time than painting a joint surface during surgery with a pointer probe.

In such variants, the computer 150 may provide a scanning workflow, e.g., via directions on a monitor, that direct the user of the 3D scanner 110 as to where to start to scan, what direction to scan in, etc., so as to gather the scan data for building the 3D model in a specific order and manner. The system 100 can then employ AI models or the like to recognize bony landmarks, fiducials, and other features to help orient and build the 3D model. Once the 3D model is built, including the fiducials, the imageless CAS navigation systems 160 can use the model to track the fiducials, e.g., via their attached tracking devices.

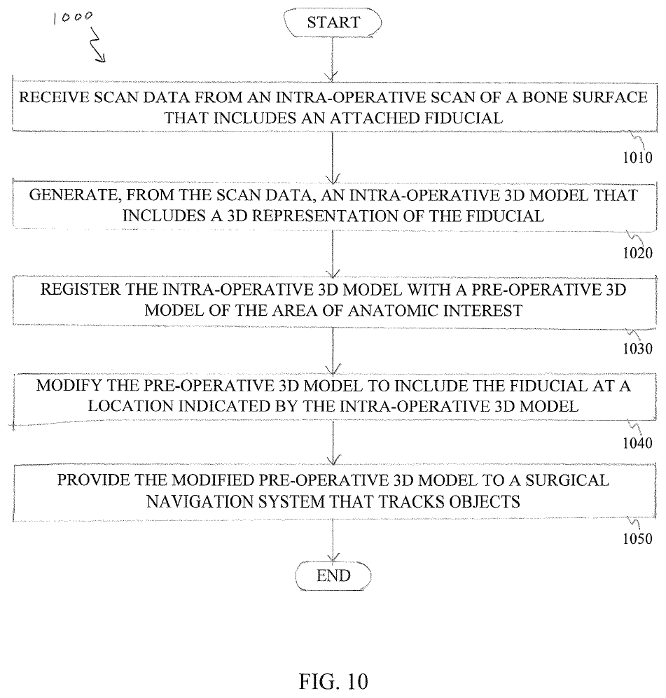

FIG. 10 is an example of a process 1000 for modifying a 3D model of a region of anatomical interest to include a fiducial in accordance with the present disclosure. In various embodiments, the process 1000 may be implemented by a computer 150 that is part of a surgical system 100, as described herein.

The process 1000 starts at block 1010 with receiving scan data from an intra-operative scan of a bone surface, where the scan data includes data representing a fiducial. In various implementations, the scan data may be intra-operatively generated by intra-operative 3D scanner, and the fiducial may have been affixed to an object, e.g., a bone, in an area of anatomic interest that is involved in a surgical operation.

At block 1020, the process 1000 continues with generating, from the scan data, an intra-operative 3D model that includes a 3D representation of the fiducial. As noted above with regard to FIG. 9, there is commercial software that may be used to generate a 3D model from scan data, including software that is associated with or comes with 3D laser scanners.

At block 1030, the process 1000 continues by registering the intra-operative 3D model with a pre-operative 3D model of the area of anatomic interest. In various implementations, this may involve overlaying and/or aligning the surfaces/features of the two models in a close manner, such as may be achieved using an iterative closest point (ICP) algorithm.

The process 1000 next modifies the pre-operative 3D model to include the fiducial at a location indicated or specified by the intra-operative 3D model, at block 1040. Thus, the pre-operative 3D model which did not include the fiducial because the fiducial was not in place prior to the surgical operation is changed to now include a representation of the fiducial. In some alternative embodiments, instead of modifying the pre-operative 3D model, the computer 150 is programmed to create a new 3D model that is a combination of the pre-operative 3D model and the intra-operative 3D model and that includes a representation of the fiducial, leaving an unchanged copy of the pre-operative 3D model stored in the system 100.

At block 1050, the process 1000 continues providing the modified pre-operative 3D model to a surgical navigation system that tracks objects, e.g., during the surgical operation. As explained above with respect to FIG. 9, the tracking is done based on the location of the fiducial, as the tracking device (e.g., tracking marker) that is attached to the fiducial using a known fixed geometry, such that tracking the location of the tracking marker reveals the location of the fiducial, which reveals the location of the object (e.g., bone, medical instrument, etc.) to which the fiducial is attached. Generally, the tracked objects are in the area of anatomic interest 140 that the surgical navigation system covers.

One of ordinary skill will recognize that the blocks, operations, functions, and details shown in FIG. 10 are examples presented for conciseness and clarity of explanation. Other blocks, operations, functions, details, and variations may be used without departing from the principles of the invention, as these examples are not intended to be limiting, and many different implementations are possible. For example, the order of the blocks may be varied, some blocks may be executed in parallel with others, the functionality of two more blocks may be combined into a single block, and/or blocks could be eliminated.

The foregoing has been a detailed description of illustrative embodiments of the invention. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for modifying a 3D model of an area of anatomic interest, the system comprising:
a fiducial that is attached to a bone surface in the area of anatomic interest;
an intra-operative three-dimensional (3D) scanner; and
a computer that is connected to the intra-operative 3D scanner and that is configured to perform a method comprising:
receiving, from the intra-operative 3D scanner, scan data from an intra-operative scan of the bone surface, wherein the scan data includes data representing the fiducial;
generating, from the scan data, an intra-operative 3D model that includes a 3D representation of the fiducial;
registering the intra-operative 3D model with a pre-operative 3D model of the area of anatomic interest;
modifying the pre-operative 3D model to include the fiducial at a location according to the intra-operative 3D model; and
providing the modified pre-operative 3D model to a surgical navigation system that tracks objects in the area of anatomic interest based on the location of the fiducial.

2. The system of claim 1, wherein the intra-operative 3D scanner is a handheld laser 3D scanner.

3. The system of claim 1, wherein the pre-operative 3D model is created from MRI scan data.

4. The system of claim 1, wherein the method further comprises:
employing a machine learning model to identify an anatomical landmark in the intra-operative 3D model.

5. The system of claim 1, wherein the fiducial is a plate or an anchor.

6. The system of claim 1, where the fiducial is attached to the bone surface at a predetermined location.

7. The system of claim 6, wherein the predetermined location is based on a preoperative image of the bone surface.

8. The system of claim 7, wherein the preoperative image is at least one of: a computer tomography image, an ultrasound image, or a magnetic resonance image.

9. The system of claim 5, wherein the plate includes a bar code the encodes surgery-assisting information.

10. The system of claim 1, wherein the method further comprises:
analyzing the intra-operative 3D model to differentiate between different types of tissue.

11. The system of claim 10, wherein the analyzing employs machine learning methods.

12. The system of claim 10, wherein the different types of tissue include bone tissue and cartilage tissue.

13. The system of claim 1, wherein:
the fiducial is attached intra-operatively without using a predetermined location on the bone surface; and
the method further comprises: analyzing the intra-operative 3D model to detect the location of the fiducial.

14. The system of claim 1, wherein registering the intra-operative 3D model with a pre-operative 3D model comprises:
aligning the intra-operative 3D model with the pre-operative 3D model using an iterative closest point (ICP) algorithm.

15. The system of claim 1, wherein modifying the pre-operative 3D model to include the fiducial at a location according to the intra-operative 3D model comprises:
combining the intra-operative 3D model with the pre-operative 3D model to produce the modified pre-operative 3D model.

16. The system of claim 15, wherein the modified pre-operative 3D model includes the fiducial from the intra-operative 3D model.

17. A computer-implemented method for modifying a 3D model of an area of anatomic interest using an intra-operative 3D scanner, the method comprising:
receiving, from the intra-operative 3D scanner, scan data from an intra-operative scan of a bone surface in the area of anatomic interest, wherein the scan data includes data representing a fiducial;
generating, from the scan data, an intra-operative 3D model that includes a 3D representation of the fiducial;
registering the intra-operative 3D model with a pre-operative 3D model of the area of anatomic interest;
modifying the pre-operative 3D model to include the fiducial at a location according to the intra-operative 3D model; and
providing the modified pre-operative 3D model to a surgical navigation system that tracks objects in the area of anatomic interest based on the location of the fiducial.

18. The method of claim 17, wherein registering the intra-operative 3D model with a pre-operative 3D model comprises:
aligning the intra-operative 3D model with the pre-operative 3D model using an iterative closest point (ICP) algorithm.

19. The method of claim 17, wherein modifying the pre-operative 3D model to include the fiducial at a location according to the intra-operative 3D model comprises:
combining the intra-operative 3D model with the pre-operative 3D model to produce the modified pre-operative 3D model.

20. The method of claim 19, wherein the modified pre-operative 3D model includes the fiducial from the intra-operative 3D model.
